(19) United States
(12) Patent Application Publication
(10) Pub. No.: US 2005/0013727 A1
(43) Pub. Date: Jan. 20, 2005

(54) System and process for removing or treating harmful biological and organic substances within an enclosure

(76) Inventor: David E. Hedman, Ojai, CA (US)

Correspondence Address: KELLY BAUERSFELD LOWRY & KELLEY, LLP, 6320 CANOGA AVENUE, SUITE 1650, WOODLAND HILLS, CA, 91367, US

(21) Appl. No.: 10/917,792

(22) Filed: Aug. 12, 2004

Related U.S. Application Data
(63) Continuation-in-part of application No. 10/371,826, filed on Feb. 20, 2003.
(63) Continuation-in-part of application No. 10/313,901, filed on Dec. 5, 2002.

(51) Int. Cl. A61L 2/06

(52) U.S. Cl. 422/3; 422/28; 422/26; 422/33; 422/24

(57) ABSTRACT

A system and process is provided for removing or treating harmful biologic and organic substances within an enclosure, such as a container, building or vehicle. Air within the enclosure is heated to a predetermined temperature to kill organisms and cause harmful substances in the structure to migrate into the ambient air. Boric acid in combination with the heat may be used to kill mold and similar undesirable organisms. Temperature and pressure levels are monitored. The heated air carrying the harmful substances is passed through a filter to remove the harmful substances, and the heated air is re-circulated to enhance efficiency. The system effectively kills insects, molds, viruses and bacteria and reduces the levels of allergens and volatile organic compounds in the structure.

### RELATED APPLICATIONS

This is a continuation-in-part of U.S. patent application Ser. No. 10/371,826, filed Feb. 20, 2003. This is also a continuation-in-part of U.S. patent application Ser. No. 10/313,901, filed Dec. 5, 2002.

### BACKGROUND OF THE INVENTION

The present invention relates to methods of sanitizing buildings, passenger occupiable vehicles, and other enclosed or enclosable spaces. More particularly, the present invention relates to a system and method for killing and removing insects, dust mites and their allergens, bacteria, viruses, fungi, molds, and volatile organic compounds from such enclosures.

A large number of methods have been developed for killing insects, such as termites, in buildings. The most widely used method is tenting the building, then filling the building with a toxic gas for a period of time sufficient to kill termites or other selected insects. This method is effective for killing termites and other insects. However, this method generally requires 12 hours to be effective, requiring building occupants to move out and businesses to be closed for approximately a three day period to insure proper venting of toxic material and/or gas. Tenting the building with heavy tarpaulins requires workers to walk and arrange the tarpaulins on the roof, often damaging the roof system. Food and medications must be placed in sealed containers or removed. Generally the entire building must be treated, even if the infestation is localized.

Techniques of varying effectiveness have been developed using heated air or very cold air to kill termites and other organisms. Typical of these are the methods disclosed by Charles Forbes in U.S. Pat. No. 4,817,329, and Hedman et al. in U.S. Pat. No. 6,327,812 in which wood destroying insects, e.g., termites, are killed by applying a heated gas, such as heated air, to wooden surfaces or the like until the core of wooden structures is heated to a temperature typically about 120° F. to 135° F. Temperatures for killing other insects are said to be surprisingly close to this range. This method has been found to be very effective for killing termites. Another alternative to the toxic gas method is disclosed by James J. Chaudoin, et al. in U.S. Pat. No. 4,958,456, in which insects, e.g., roaches, fleas and beetles, are killed by a treatment of building spaces with boric acid and heat. However, the methods disclosed in the Forbes and Hedman et al. patents are quite complex in the preparation of the building. An enclosing tent structure must be formed around the structure to be decontaminated. Tenting the building with heavy tarpaulins requires workers to walk and arrange the tarpaulins on the roof, often damaging the roof system.

Also, these methods, using the described temperatures, is not effective for other organisms, such as fungi, and molds such as, but not limited to, aspergillus oryzae, aspergillus terreus, aspergillus versicolor, cladosporium hergbarum, stachybotrys chartarum, penicillium aurantiogriseum, pencillium chrsogenum, pencillium gladrum and fusarium oxysporum. Further, many such fungi, molds and the like are a serious health hazard even when dead. Many people are allergic to the dust-like remains and residue, i.e., allergens, of these organisms that can also cause serious health problems. This is a particular problem to persons suffering from asthma, bronchitis, pneumoconious and other respiratory ailments, and is a common contributing factor to sick building syndrome (SBS).

It is also well-known that the heated air causes certain molds, fungi, etc. to sporulate, thus releasing spores into the structure and thus dispersing the harmful biological agents and possibly contaminating the structure to a greater degree than originally presented. The use of positive pressure within the structure, as described in Forbes and Hedman et al., further increase the likelihood that the biological contaminants will be dispersed throughout the structure. Forbes and Hedman et al. also disclose that the heated air can be vented from open windows and the like. However, when treating a contaminated building having harmful viruses, toxic molds, etc., it is not desirable to release such contagions into the air.

Volatile organic compounds (VOCs) have also been implicated as a possible cause of SBS. VOCs can originate from a variety of sources. Commercial examples include by-products of printing shop operations, office machine repairs, blueprint production, photographic processing and food service operations. In residences, such VOCs can include hobbyist products, cosmetics, perfumes, personal hygiene products, aerosol sprays, tobacco smoke, pet urine and even small emissions from the bodies of the occupants. Off gassing of VOCs is often a common by-product of various building/construction materials, for example paints, adhesives, plastics, carpeting, etc.

Such VOCs are implicated with SBS for mostly two reasons. First, the health effects from exposure to VOCs are consistent with SBS, ranging from irritant effects such as unpleasant odors and mucous membrane irritation, through general systemic effects such as fatigue, nausea, and difficulty concentrating. In addition, they may be of importance because some of them have been shown to have carcinogenic or adverse reproductive effects. Second, indoor concentrations of VOCs, particularly in new buildings, are often greatly elevated with respect to outdoor VOC concentrations. In fact, indoor VOC concentrations have typically been found to be two to ten times higher then outdoor concentrations, and indoor concentrations as much as 100 times higher than outdoor concentrations have been reported in new buildings.

Passenger occupiable vehicles, such as trains, buses, airplanes, etc. also include building/construction materials which are known to off-gas VOCs. Also, the fuel, oil, and grease fumes and odors can infiltrate the passenger compartments of such vehicles and build-up within the seats, carpets, etc. over time. Due to the great number of people regularly traveling in such vehicles, there is an increased chance of coming into contact with contagious bacterium or viruses that can cause illness. Other organisms, such as fungi, and toxic molds can also be potentially found in such vehicles. As the company owning such vehicles necessarily must keep the vehicles running nearly constantly in order to realize the expected profit, such vehicles are rarely cleaned thoroughly. Even if the surfaces are superficially vacuumed and wiped down, there still remain live and dead organisms such as lice, mites, fungi, toxic molds, bacterium, viruses, VOCs, oxidized odors, and potentially insects which may have infested the vehicle, particularly those where food is prepared or served.

There are also instances when personal articles and small pieces of furniture need to be treated. For example, bedding and mattresses over time can accumulate a large amount of allergens, in the form of dust mites and their allergens, etc. Furniture may also experience water damage, causing fungi and toxic molds to grow thereon. These articles may also need to be treated for contagious bacterium or viruses that can cause illnesses.

Accordingly, there is a need for a system and method for killing and removing biological organisms and reducing odors and volatile organic compounds in enclosures such as commercial and residential buildings, boats, vehicles and portable containers. Such a method should be non-toxic and performed in a relatively short amount of time. Such a method should also effectively kill and remove a large proportion of the dead organisms and substantially reduce volatile organic compounds. The present invention fulfills these needs and provides other related advantages.

### SUMMARY OF THE INVENTION

The present invention resides in a system which removes or treats harmful organic substances within an enclosure, such as a building, vehicle, container, erectable enclosure or other enclosed structure. Such organic substances can include VOCS, and the process of the present invention also allows to killing and removing harmful biological substances and organisms, such as lice, mites, fungi, toxic mold, bacterium, viruses, and insects. Collectively, such harmful biological organisms and substances are referred to as biotics.

Larger structures are typically prepared by positioning a plurality of probes, such as temperature probes and pressure measuring devices, at predetermined locations within the structure. Alternatively, other means for determining the temperature within the structure are used. For example, in a particularly preferred embodiment, thermal imaging using an infrared camera and imaging software is used to determine the internal temperatures of the structure during the process of the present invention.

Heat-sensitive articles within the structure are protected. This can be done by covering the articles with an insulated mat. Also, fans can be positioned adjacent to the heat sensitive articles for directing the flow of heated air away from the articles during the decontamination process.

The contaminated area of the structure may also be physically cleaned in preparation of decontamination. This can be done by wiping, scraping, vacuuming, etc. the mold or other harmful organisms which are accessible and can be easily cleaned and removed. In one embodiment, borate, such as boric acid or the like, is applied to selected areas of the enclosure to kill molds and fungi.

The ambient air within the structure is then heated to a predetermined temperature of between 110° F. and 400° F., typically by directing and distributing heated air into the enclosed structure. This causes the harmful substances in the structure to be destroyed, neutralized, oxidized, or migrate into the ambient air. A biocide or even moisture; may be added to the heated air to enhance the treatment. Preferably, the air within the structure is aggressively moved using blowers, fans, or the like to aerosolize the biological and organic substances to facilitate their removal. The temperature of the structure is monitored until the predetermined temperature is achieved. The pressure levels within the structure are also monitored to verify adequate pressure, which is typically a negative pressure to facilitate removal of the harmful substances.

The heated air carrying the harmful substances is then removed from the structure through a filter. The filter preferably comprises a high efficiency particulate arrestance filter. Preferably, the filtered and heated air is re-circulated into the enclosure. In a particularly preferred embodiment, after a predetermined time period of directing heated air within the structure, the non-heated filtered ambient air is directed into the structure, while continuing to remove the air through the filter. In certain instances, the contaminated portion of the structure is then physically cleaned after these steps have been performed.

Other features and advantages of the present invention will become apparent from the following more detailed description, taken in conjunction with the accompanying drawings, which illustrate, by way of example, the principles of the invention.

### BRIEF DESCRIPTION OF THE DRAWINGS

The accompanying drawings illustrate the invention. In such drawings:

FIG. 1 is a schematic diagram showing components of the system of the present invention installed for treatment of a building;

FIG. 2 is a schematic diagram showing an automobile treated in accordance with the present invention;

FIG. 3 is a schematic diagram illustrating a train being treated in accordance with the present invention;

FIG. 4 is a schematic diagram of an airplane being treated in accordance with the present invention;

FIG. 5 is a schematic diagram illustrating a portable decontamination system used in accordance with the present invention to treat smaller objects; and

FIGS. 6A and 6B are flow diagrams illustrating the method used in accordance with the present invention;

### DETAILED DESCRIPTION OF THE PREFERRED EMBODIMENTS

As shown in the accompanying drawings for purposes of illustration, the present invention is related to a system and method for removing harmful organic substances, such as VOCs, dust mites and their allergens, bacteria, termites and other insects, from an enclosure.

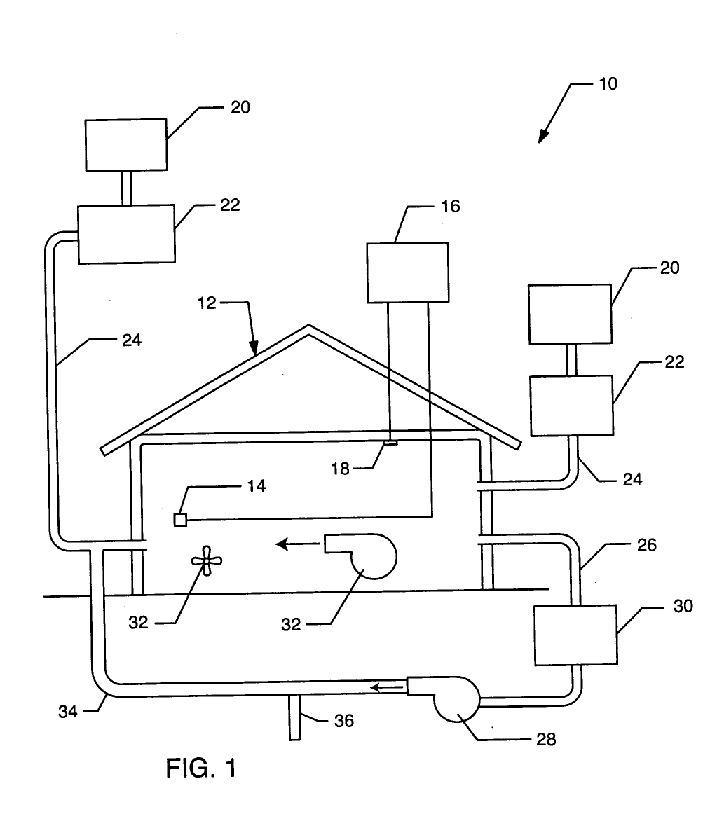

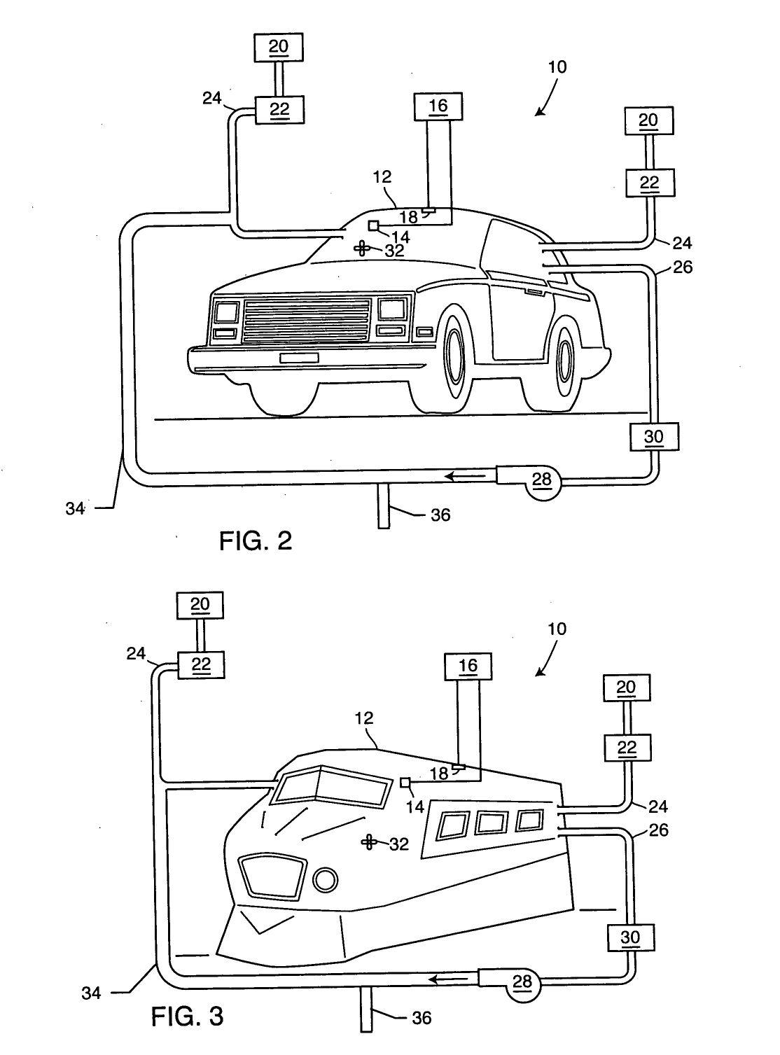

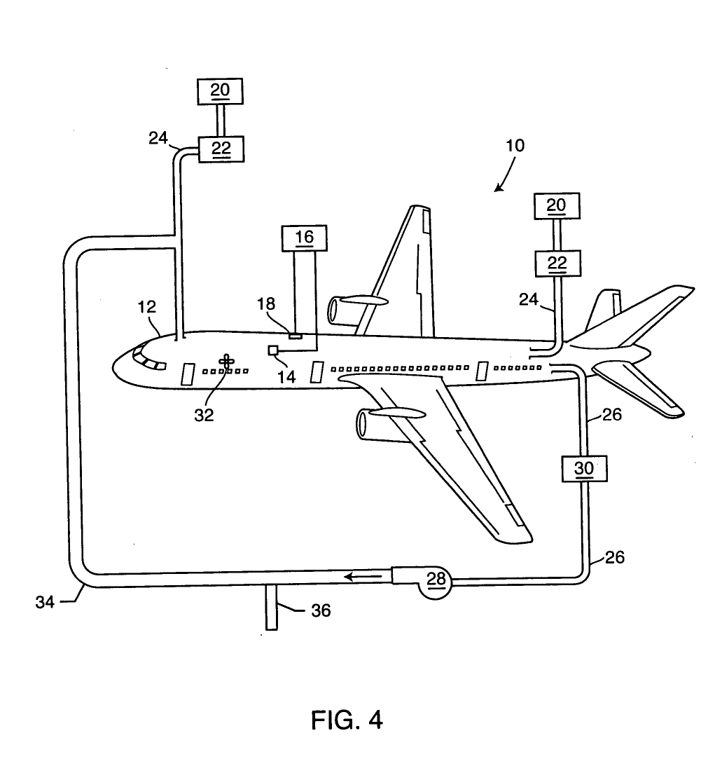

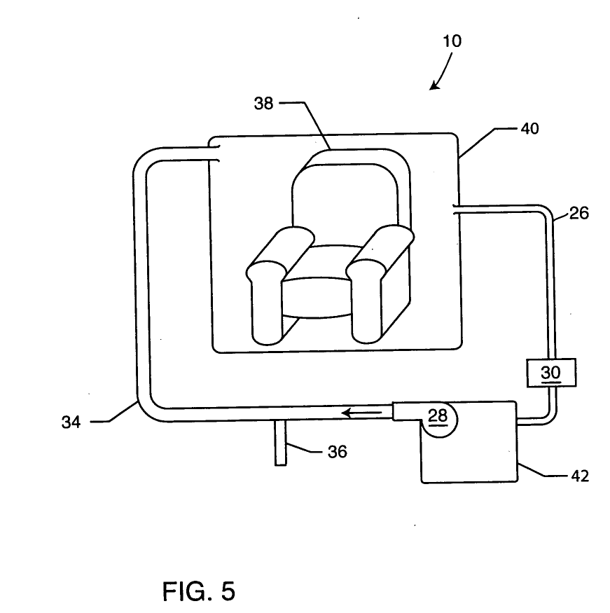

Referring to FIGS. 1-4, there is seen a schematic diagram showing the components of the system of the present invention, referred to generally by the reference number 10, in use treating an enclosed structure 12. The enclosed structure 12 is typically a commercial or residential building, but can also be a vehicle, such as an airplane, bus, boat, automobile, etc., as shown in FIGS. 2-4.

A plurality of temperature sensors 14 are positioned at predetermined locations within the structure to monitor the temperature of the structure 12. Typically, these sensors 14 have thin, elongated tips that can be adhered to or pushed into materials to be heated or into suitably sized holes drilled into such materials so as to measure the surface and/or internal temperature. The sensors 14 may be wired to a console 16 which displays and records the temperature at each sensor 16 in real time. Alternatively, the sensors 14 may be wireless and transmit a signal to the console 16. Typical sensors 14, as for way of example and not by way of limiting, include thermal couples, thermistors, or the like connected to a computer and/or a strip chart recorder console 16.

In a particularly preferred embodiment, instead of utilizing temperature sensors within the structure 12, thermograpy is used. This entails utilizing a specialized infrared camera which detects the thermal and infrared energy emitted by the structure during the treatment of the present invention. Specialized software coupled with the camera enables the operator to “see” and measure thermal energy emitted from the structure 12. This allows non-contact temperature measurement of the structure 12. The software and camera can be set to a given temperature range such that when the structure is within the temperature range, the resulting graphic image indicates such. FLIR Systems, Inc. provides such cameras and infrared cameras and software which can be utilized by the present invention. Utilizing the infrared thermal imaging system saves in the labor cost associated with placing and wiring the temperature sensors.

A pressure measuring device, such as a manometer 18, is positioned within the structure 12 so as to measure the internal pressure of the structure 12 during operation of the invention. As will be more fully described herein, in some instances, positive air pressure is desirable. However, in most instances, a negative pressure is established and maintained throughout the operation of the method of the present invention in order to prevent the dispersal of harmful biological and organic contaminants throughout the structure 12. The manometer 18 can be linked to the console 16 to provide the pressure information from without the structure 12.

One or more heaters 20 heat air to a predetermined temperature lethal to the organisms to be destroyed. For a more complete disinfection, the air temperature is preferably raised to at least about 155° F., with optimum results generally achieved with temperatures in the range of about 110° F. to 400° F., or higher. A biocide, having desirable characteristics enhanced by heat, may be introduced with the heated air. Moisture may also be introduced with the heated air.

Any suitable heater 20 may be used. A gas burning heating device 20, such as a conventional propane heater, is preferred as being particularly efficient in heating air. Any other heating arrangement, such as electrical devices, solar heaters, and light emitting devices, may be used if desired. Preferably, a filter is placed at the air ingress of the heater 20.

The air can be heated directly within the structure 12, however, the heated air is usually injected into the structure 12. Heated air (and biocide, if used) from the one or more heaters 20 is directed through a blower 22 (which may, if desired, be a component of the heater 20) which injects the hot air into the enclosed structure 12 through at least one inlet duct 24. Generally, a plurality of inlet ducts 24 will be used to achieve the optimum distribution of hot air throughout the enclosed structure 12. The inlet ducts 24 preferably include variable flow dampers and may be moved while the system is in operation to achieve uniform temperatures in all areas of the structure being treated, as sensed by sensors 14 and observed at a console 16.

At least one outlet duct 26 is provided to allow the air to be removed from the structure 12. A blower or vacuum 28 is connected to the outlet duct 26 in order to remove air from the interior of the structure 12. The vacuum 28 may be used to create a negative pressure within the structure 12. Typically, this negative pressure is created before the heated air is introduced into the structure 12. The removed air is filtered, typically utilizing a high particulate arrestance filter, ULPA filter, or the like coupled with the vacuum/blower 28. Other filters such as charcoal filters or UV filters may be employed as well. The filter or air scrubber 30 removes the remains of the organisms and VOCs from the air to prevent them from reaching the environment or being re-introduced into the structure 12. Of course, such filters are also suitable for use in connection with the ingress of the heater 20.

Preferably, additional blowers 32 or fans are positioned within the structure 12 to aggressively move the air within the structure to further enhance the removal of harmful biotics and organic substances by aerosolizing the biological and organic substances and aid in heat distribution. Additionally, fans 32 may be positioned strategically within the structure 12 to selectively move the air away from predetermined heat-sensitive articles or areas of the structure in which such an elevated temperature is not desired. Typically, however, such heat-sensitive articles are removed from the structure or covered with insulation mats or the like.

In a particularly preferred embodiment, the filtered air is re-directed through a duct 34 into the structure 12, such as by linking the duct 34 with the inlet 24. Such re-circulation of heated air enhances the energy and thermal efficiency of the process and decreases the overall treatment time. Additionally, it has been found that merely venting the air into the environment causes heat dilution and stratification to occur within the structure 12. Re-circulating the filtered and heated air reduces the heat dilution and stratification, and has been found to increase treatment air circulation within the containment area of the structure 12. The re-circulated air may be blended with the heat processed air as it exits the heater barrel, re-heated by the heater 20 or simply re-introduced by way of ducting into the structure 12.

Although the above description has been directed to rather large structures, such as residential or commercial buildings (FIG. 1), and passenger occupiable vehicles (FIGS. 2-4), and the like, the present invention can also be applied to treatment of much smaller areas or objects. For example, a single room of a building may be treated by sealing the windows, doors, and other passageways of that particular room or area and treating such area, as described above. There are also instances where small personal articles, such as clothing or bedding, or even furniture is required to be treated, but not the structure itself.

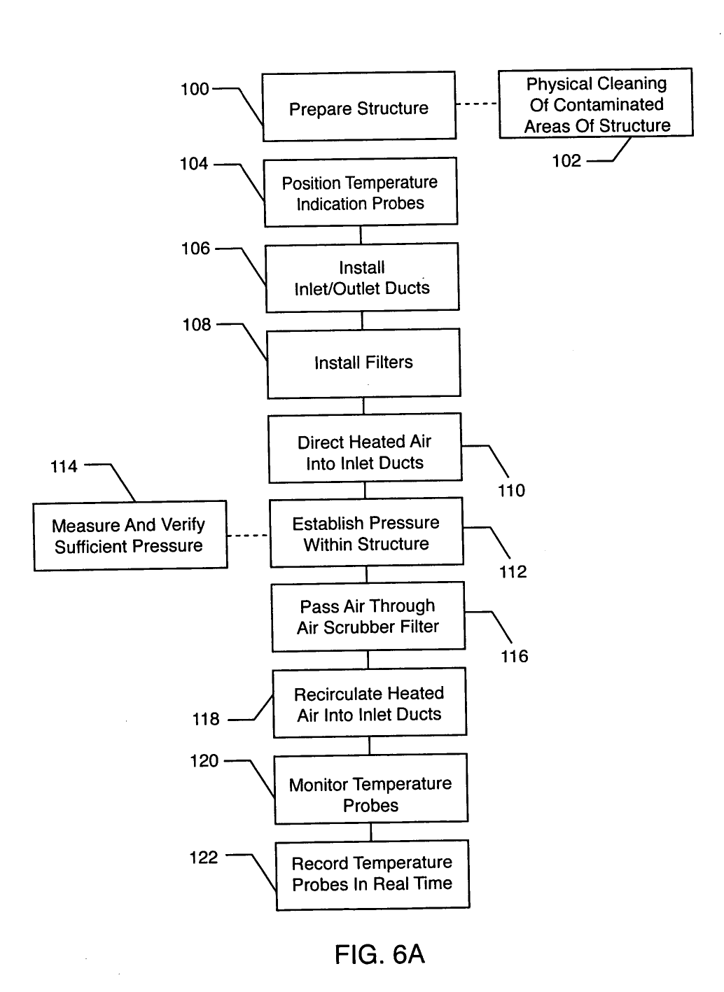

As illustrated in FIG. 5, the present invention can be adapted for treatment of such articles 38. A common instance of treatment is the destruction or removal of allergens such as dust mite feces and the like from bedding and mattresses. Dust mite feces are known to cause mild and even severe allergic reactions in some individuals. These individuals may have headaches, runny noses, persistent coughs, etc. which is not caused by an infection, but rather allergic reaction to the allergens. The personal articles 38, in the form of bedding or the like, is placed within a portable structure 40. Such portable structure 40 may comprise a rigid and portable structure of sufficient size to treat the articles. For example, the back of a van may be converted into a treatment containment area. Alternatively, an inflatable bag, typically comprised of appropriate thermal material, is used. The personal articles 38 would be placed within such a thermal envelope or bag 40 and heated air directed into the inlet thereof. Pressure and temperature could be monitored and controlled using a device 42 attached to the portable structure 40. Preferably, the heated air which is removed is passed through a filter 30 and re-circulated, as described above. If toxic molds or fungi are of a concern, the air temperature may be reduced over time to prevent sporulation and the like.

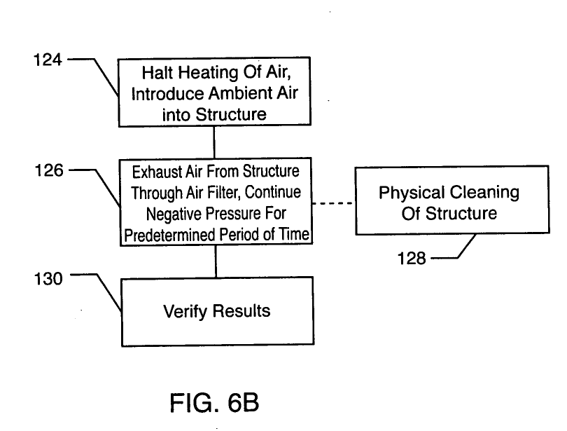

With reference to FIG. 6A, in the operation of the system of the invention, the first step is to prepare the structure, as indicated in block (100). This basically involves removing all heat-sensitive items from the enclosure or, in some cases, covering heat sensitive items, such as electronic devices and plastic items, with thermal insulation material. All material that has a flash/melt point at or below the maximum temperature to be used (such as candles, lipstick, etc.) must be removed.

Typically, the preparation of the structure also includes physical cleaning of contaminated areas of the structure (102), which may be preformed while the area is under a negative pressure. This can include vacuuming, wiping, scraping, etc. of various surfaces which have been contaminated with harmful biological contaminants, such as mold, fungi or bird, rodent or insect debris, etc. In extreme cases, this may require the removal of carpeting, section of walls, etc. However, the invention is intended to neutralize and remove these biological and organic contaminants without requiring resort to such extreme measures in some instances.

In one embodiment, particularly when treating the structure 12 for mold and fungi, borates, and preferably boric acid, are dispersed within the structure 12 at locations, preferably, where mold and fungi are likely to be encountered. Boric acid, H₃BO₃, is a white crystalline, oxygen-bearing acid of boron found in certain minerals and volcanic waters or hot springs in certain mineral deposits. Boric acid, or salts of boric acid, borates, traces of boron are necessary for growth of land plants and thus are indirectly essential for human life. In excessive quantities, however, borates may act as unselective herbicides. The most common source of boric acid is borate, sodium tetraborate or borax, which occurs naturally in salt beds. Boric acid may be obtained by treating borate with sulfuric acid. Boric acid is commonly used as a mild antiseptic for burns and surface wounds and comprises a major ingredient in eye lotions. Among its other important applications is its use as a fire retardant in fabrics. Importantly, boric acid is non-toxic to humans and animals and is ecologically benign in low concentrations.

Applying boric acid using conventional applicator methods and devices, i.e., dusting boric acid as a conventional insecticide as dust, spraying a solution or slurry or dispersion of boric acid, etc., coupled with heating the air within the enclosure, advantageously improves mold, fungi and pest (termite) abatement within the structure 12. The borates may be used in pre-treating contents of an enclosure, such as building materials, lumber, etc. or in post-treating such contents after application of heat.

A plurality of temperature indicating and pressure measuring probes 14 and 18 are placed in predetermined locations as indicated in block (104) to assure that the required temperature levels are achieved. In some cases the probes 14 can be read directly, although preferably they are connected by wires or wireless means to the console 16, so that all probes 14 and 18 can be monitored conveniently and the data recorded in real time. Alternatively, as discussed above, thermal imaging cameras and software may be used instead of the temperature probes 14.

When the enclosed structure 12 is sealed, at least one inlet duct 24 and at least one outlet duct 26 are then installed as indicated in block (106). Generally, a plurality of inlet ducts 24 is preferred. Although each duct 24 may enter the enclosed structure 12 separately, the use of one inlet duct 24 connected to a manifold from which plural ducts extend to predetermined locations within the enclosed structure 12 is preferred. Ducts 24 may enter the structure 12 through any suitable opening, such as an open window or door with the remainder of the window or door blocked by a panel. In some instances, such as when treating vehicles, tenting may actually be required or desired to treat the structure 12. However, in most instances such tenting is not required.

The appropriate air scrubbing filters 30 and vacuum devices 28 for facilitating the removal of the heated air and filtering the harmful substances therefrom, is installed, as indicated in block (108).

When the components of the system 10 have been properly prepared and positioned, heated filtered air is directed into the inlet ducts (110). The desired pressure is established within the structure 12 (112) and the manometer or other pressure sensing device is used to verify that a sufficient pressure is present (114). In some instances, a positive pressure is actually desired wherein the ingress of heated airflow into the containment area exceeds the egress air flow from the negative air machines 28. Such positive pressure may be desired to force the contaminants to aerosolize or otherwise enter the circulated air. Typically, a negative air pressure within the structure 12 is desirable, by removing air more quickly than it is introduced, to ensure the removable of the contaminants therefrom and to promote circulation of the air. In any event, a negative pressure will be applied to the structure 12 at some point of the process in order to remove the aerosolized contaminants and filter them. This is accomplished using the vacuum/blower device 28 and filter 30 as described above. Using the pressure measuring manometer device 18, the internal pressure of the structure is measured and it is verified that sufficient negative pressure is present (114). Often the establishment of negative pressure is performed before any heat is introduced into the structure in order to begin the removal of any loose and aerosolized contaminants, and prevent their sporulation before heat is introduced.

The heated air is then re-circulated into the inlet ducts (118). Flow of the heated air through the enclosed structure 12 may range in time from a few hours to several days to provide optimum results. During this time, the temperature probes 14 are monitored (120) and these results recorded in real time (122) to ensure that the intended areas within the structure 12 are properly treated.

The heated air which has been circulated through the structure 12 is continually removed through an air scrubber filter to remove the remains of the destroyed organisms and VOCs. Biocides, such as ozone, or even moisture may be added to the heated air to enhance the treatment effect.

At any time during system operation, the inlet and outlet ducts 24 and 26 may be moved to assure uniform temperatures throughout the structure, as indicated by the temperature probes 14 and temperature monitoring console 16.

With reference now to FIG. 6B, after a predetermined period of time in which it has been determined that the harmful biological organisms and agents have been destroyed, moisture, if previously introduced is removed from the heated airstream, and after sufficient drying has taken place, the heating of air is halted and non-heated ambient air is introduced into the structure (124). The air from the structure is then exhausted through the air filter while the negative pressure is maintained for a predetermined period of time (126). These steps are taken in order to prevent any viable fungi, molds, etc. from sporulating or the like as such organisms when threatened with destruction will often sporulate or form cysts or the like to facilitate the survival of the organisms and their progeny. The aggressive air flow through the structure continues to remove the harmful organisms, organic substances, etc., for some time.

This entire process may often be completed in five to twelve hours, for example, allowing a business to be closed for only one day or a residential structure to be fully treated during a typical work or school day. However, in certain circumstances, such as in the case of large structures or high levels of harmful substances within the structure, the process may be extended to several days or more to ensure that the structure is properly treated. It has been found that while harmful organisms are killed and removed during this process, the reduction of the VOCs actually continues for some time after treatment. Placing a filtering system within the structure and/or opening a window to allow the structure 12 to properly vent is believed to be adequate to remove these residual compounds.

In certain instances, the structure 12 is then physically cleaned (128) after the aforementioned steps have been performed. For example, when dealing with the hanta virus, the health concerns of the workers dictate that the virus be killed and removed to the greatest extent possible. Then, after the virus has been destroyed and removed to the greatest extent possible utilizing the aforementioned steps, workers can enter the structure and physically remove rodent droppings and the like which may contain the neutralized viruses. Samples and specimens may be taken of the previously contaminated areas to verify the desired results (130) and a physical examination of the structure can be used to verify the removal of the contagions and harmful substances.

It will be apparent to those in the art that the present invention can be adapted to other types of enclosures as well. For example, the invention can be adapted to the cleansing and disinfecting of unusual structures such as swimming pools. In this regard, acid washing of swimming pools is a common practice when the pool builds up a black fungi. The common practice is to drain the pool and then bathe the walls of the pool with an acid wash which kills the fungi. A less toxic approach utilizing the present invention would be to drain the pool and then cover it with thermal blankets to trap heat. The thermal blankets and the pool would define the enclosure. The enclosure would then be heated to a temperature which is lethal to the fungi. In this situation, all of the processes described above would be applicable.

With regard to the use of biocides, it has been found that by adding sea water to wood or paper, and then heating the solution for penetration, it is possible to prevent microbial growth. The addition of heat in connection with the process of the present invention aids in penetration of the mineralized sea water to, essentially, fossilize wood base products. Moreover, while the foregoing describes the use of biocides added to the heated air, it will be understood that the biocides may be applied before, during or after the heating process so that the biocides may act synergistically with the heat.

Although several embodiments have been described in detail for purposes of illustration, various modifications may be made without departing from scope and spirit of the invention. Accordingly, the invention is not to be limited, except as by appended claims.

1. A process for removing or treating harmful biological and organic substances within an enclosure, comprising the steps of:
heating ambient air within the enclosure to a predetermined temperature to cause the harmful substances in the enclosure to be destroyed or migrate into the ambient air;
utilizing a thermal imaging device to monitor the temperature in the enclosure until the predetermined temperature is achieved.

2. The process of claim 1, including the step of removing the heated air carrying the harmful substances from the enclosure through a filter.

3. The process of claim 1, including the step of recirculating the filtered and heated air into the structure.

4. The process of claim 1, including the step of physically cleaning a contaminated portion of the enclosure to prepare the structure.

5. The process of claim 1, including the step of physically cleaning a contaminated portion within the enclosure after determining that adequate treatment has occurred.

6. The process of claim 1, including the step of placing and distributing heated air into the enclosure.

7. The process of claim 6, including the step of introducing moisture into the heated air.

8. The process of claim 6, including introducing a biocide within the enclosure.

9. The process of claim 1, including the step of applying borate to selected areas of the enclosure.

10. The process of claim 1, wherein the filter comprises a high or ultra-high efficiency particulate arrestance filter.

11. The process of claim 1, wherein the filter comprises a carbon filter.

12. The process of claim 1, including the step of protecting heat-sensitive articles within the enclosure.

13. The process of claim 12, including the step of covering the articles with an insulated mat.

14. The process of claim 12, including the step of directing the flow of heated air away from the heat sensitive articles.

15. The process of claim 1, including the step of establishing a pressure within the enclosure and monitoring the pressure within the enclosure.

16. The process of claim 15, including the step of positioning a manometer within the structure to measure the pressure within the enclosure.

17. The process of claim 15, including the step of creating a negative pressure within the enclosure and monitoring pressure levels within the enclosure to verify adequate negative pressure.

18. The process of claim 1, including the step of aggressively moving air within the enclosure to aerosolize the biological and organic substances to facilitate their removal.

19. The process of claim 1, including the step of positioning at least one temperature probe at a predetermined location relative to the enclosure.

20. The process of claim 1, including the step of directing non-heated ambient air into the enclosure after a predetermined time of directing heated air into the enclosure, while continuing to remove the air through the filter.

21. The process of claim 1, wherein the ambient air within the enclosure is heated to between 110° F. and 400° F.

22. The process of claim 1, wherein the enclosure comprises at least a portion of a building.

23. The process of claim 1, wherein the enclosure comprises a vehicle.

24. The process of claim 1, wherein the enclosure comprises a portable enclosure.

25. The process of claim 1, including the step or irradiating contents within the enclosure with ultra-violet light.

26. The process of claim 1, including the step of irradiating the heated air with ultra-violet light.

27. The process of claim 1, including the step of filtering non-heated air introduced into the enclosure.

28. A process for removing or treating harmful biological and organic substances within an enclosure, comprising the steps of:
introducing filtered and heated air of a predetermined temperature into the enclosure to cause the harmful substances in the enclosure to be destroyed and/or migrate into the ambient air;
establishing a selected pressure within the enclosure;
monitoring the temperature in the enclosure until the predetermined temperature is achieved;
utilizing a pressure monitoring device to monitor the pressure within the enclosure;
aggressively moving air within the enclosure to aerosolize the harmful substances to facilitate their removal;
removing the heated air carrying the harmful substances from the enclosure through a filter; and
recirculating the filtered and heated air into the structure.

29. The process of claim 28, including the step of physically cleaning a contaminated portion of the enclosure to prepare the structure.

30. The process of claim 28, including the step of physically cleaning a contaminated portion within the enclosure after determining that adequate treatment has occurred.

31. The process of claim 28, including the step of introducing moisture or a biocide into the heated air.

32. The process of claim 28, including the step of applying borate to selected areas within the enclosure.

33. The process of claim 28, including the step of protecting heat-sensitive articles within the enclosure.

34. The process of claim 28, including the step of directing the flow of heated air away from the heat sensitive articles.

35. The process of claim 28, including the step of creating a negative pressure within the enclosure and monitoring pressure levels within the enclosure to verify adequate negative pressure.

36. The process of claim 28, including the step of positioning a single temperature probe at a predetermined location relative to the enclosure.

37. The process of claim 28, including the step of directing non-heated ambient air into the enclosure after a predetermined time of directing heated air into the enclosure, while continuing to remove the air through the filter.

38. The process of claim 28, wherein the ambient air within the enclosure is heated to between 110° F. and 400° F.

39. The process of claim 28, wherein the enclosure is selected from the group consisting of at least a portion of a building, a vehicle and a portable enclosure.

40. The process of claim 28, wherein the monitoring step includes the step of utilizing a thermal imaging device.

41. A process for removing or treating harmful biological and organic substances within an enclosed structure, comprising the steps of:
positioning a pressure measuring device within the structure to measure the pressure within the structure;
creating a negative pressure within the structure;
directing filtered and heated air into the structure and distributing the heated air until an interior of the structure rises to a predetermined temperature of between 110° F. and 400° F. to cause the harmful substances in the structure to be destroyed or migrate into the ambient air;
monitoring the temperature of the structure until the predetermined temperature is achieved;
monitoring pressure levels within the structure to verify adequate negative pressure;
aggressively moving the air within the structure;
removing the heated air from the structure through a filter;
recirculating the heated and filtered air into the structure;
after a predetermined period of time, directing non-heated air into the structure; and
removing the non-heated air from the structure through the filter.

42. The process of claim 41, including the step of physically cleaning a contaminated portion of the structure to prepare the structure.

43. The process of claim 41, including the step of physically cleaning a contaminated portion of the structure after it is determined that adequate treatment has occurred.

44. The process of claim 41, including the step of protecting heat-sensitive articles within the structure.

45. The process of claim 41, including the step of introducing moisture or a biocide into the enclosure.

46. The process of claim 41, including the step of applying borate to selected areas of the enclosure.

47. The process of claim 41, wherein the enclosed structure comprises at least a portion of a building or a vehicle.

48. The process of claim 41, wherein the monitoring step includes the step of utilizing a thermal imaging device.
